(19) United States
(12) Patent Application Publication
(10) Pub. No.: US 2010/0176053 A1
(43) Pub. Date: Jul. 15, 2010

(54) MATERIALS FOR REMOVING CONTAMINANTS FROM FLUIDS USING SUPPORTS WITH BIOLOGICALLY-DERIVED FUNCTIONALIZED GROUPS AND METHODS OF FORMING AND USING THE SAME

(75) Inventors: D. Jack Adams, Park City, UT (US); Jan D. Miller, Salt Lake City, UT (US)

Correspondence Address: THORPE NORTH & WESTERN, LLP., P.O. Box 1219, SANDY, UT, 84091-1219, US

(73) Assignee: UNIVERSITY OF UTAH RESEARCH FOUNDATION

(21) Appl. No.: 12/593,582

(22) PCT Filed: Mar. 28, 2008
(86) PCT No.: PCT/US2008/058682

§ 371 Date: Mar. 29, 2010

Related U.S. Application Data
(60) Provisional application No. 60/921,118, filed on Mar. 29, 2007.

(51) Int. Cl. C02F 3/04; C12N 11/08; C08G 63/91; C07K 2/00; C07K 14/00; C12N 11/14; C12N 11/02; C12N 11/10; C08B 37/04; C08B 37/00; C01B 31/08

(52) U.S. Cl. 210/614; 525/50; 525/54.1; 435/180; 525/418; 525/54.2; 525/54.24; 530/300; 530/350; 435/176; 435/177; 435/178; 536/3; 536/102; 536/56; 210/615; 502/416

(57) ABSTRACT

A modified bioreactor support material having high surface area for removing a contaminant (16) from fluids can include a substrate (10) having a functionalized surface, The functionalized surface can have inorganic or organic non-living functional groups, such that the functional groups bind to or chemically alter the contaminant. A method for making a modified bioreactor support material can include activating a suitable substrate (10) and attaching a biologically-derived functional group carrier such as living microbes (18) or non-living materials (14) derived from living materials to the activated substrate (10).

### RELATED APPLICATION

This application is related to U.S. Provisional Application No. 60/921,118, filed Mar. 29, 2007 which is incorporated herein by reference.

### BACKGROUND OF THE INVENTION

Removal and detection of contaminants from wastewater or other water sources continue to be a focus of research and a significant challenge. Economic reduction of trace contaminants and other metals from wastewater becomes especially difficult when concentrations of the contaminant are low and where water volumes are high. Standards for contaminants removal in water treatment in the United States for chemical plants, mining, culinary, and agriculture continue to increase, while many other countries throughout the world struggle with poor water quality in terms of both culinary and industrial applications. Current costs associated with wastewater treatment exceed $200 million annually, with untold negative health effects resulting in unnecessary deaths and associated socioeconomic impacts.

A large variety of approaches has and continues to be explored and developed to improve water treatment. Some of these approaches include ion exchange resins, activated carbons, reverse osmosis membranes, solvent extraction, selective membranes, and the like. Some technologies also utilize iron as a removal mechanism relying on iron oxyhydroxides. However, this technology tends to have a finite number of binding sites which when filled allow for arsenic or other contaminants to pass through. Furthermore, most of the conventional approaches tend to plug and design considerations lead to significant channeling and bypass of treatment sites within the system.

For this and other reasons, the need remains for development of methods and systems to effectively and economically remove contaminants from water or other fluids.

### SUMMARY OF THE INVENTION

In accordance with the present invention, a modified bioreactor support material having high surface area for removing a contaminant from fluids can include a substrate having a functionalized surface. The functionalized surface can have biologically-derived functional groups, such that the functional groups bind to or chemically alter the contaminant.

In accordance with another aspect of the present invention, a method for making a modified bioreactor support material can include activating a suitable substrate to have a preferential binding for functional groups and/or to have a predisposition for contaminant binding prior to attaching the functional groups thereto to expose binding sites. The biologically-derived functional groups can be attached to the activated substrate. This combined matrix of activated surface and functional groups (which can include living or non-living functional group carriers) is capable of binding, transforming or otherwise interacting to a greater extent with system contaminants during use, removing them from the system. Porous substrates and especially mesoporous substrates are currently preferred although non-porous substrates can also be useful.

Additional features and advantages of the invention will be apparent from the following detailed description, which illustrates, by way of example, features of the invention.

### BRIEF DESCRIPTION OF THE DRAWINGS

FIG. 1 is an illustration of several metal binding mechanisms in accordance with one embodiment of the present invention.

FIG. 2 is an illustration of a support matrix acting as a substrate for a combination of several different living and non-living functional groups in accordance with embodiments of the present invention.

FIG. 3 is a graph of cyanide and selenium concentration for several support materials after 18 hour retention time in accordance with several embodiments of the present invention.

FIG. 4 is a graph of enzymatic and live cell biooxidation of cyanide in accordance with an embodiment of the present invention.

FIG. 5 is a graph of contaminant removal over time for several support materials in accordance with another embodiment of the present invention.

FIG. 6 is a graph of metal loading for an example in accordance with another embodiment of the present invention.

FIG. 7 is a graph of arsenic removal over time for conventional material and support materials in accordance with another embodiment of the present invention.

FIG. 8 is a graph of enzymatic biooxidation of cyanide over time for several conventional and support materials in accordance with another embodiment of the present invention.

FIG. 9 is a graph of As and Se removal over time in accordance with another embodiment of the present invention.

FIG. 10 is a graph of contaminant loading in accordance with another embodiment of the present invention.

It should be noted that the figures are merely exemplary of several embodiments of the present invention and no limitations on the scope of the present invention are intended thereby. Further, the figures are generally not drawn to scale, but are drafted for purposes of convenience and clarity in illustrating various aspects of the invention.

### DETAILED DESCRIPTION

Reference will now be made to exemplary embodiments and specific language will be used herein to describe the same. It will nevertheless be understood that no limitation of the scope of the invention is thereby intended. Alterations and further modifications of the inventive features described herein, and additional applications of the principles of the invention as described herein, which would occur to one skilled in the relevant art and having possession of this disclosure, are to be considered within the scope of the invention. Further, before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

### DEFINITIONS

In describing and claiming the present invention, the following terminology will be used.

The singular forms “a,” “an,” and “the” include plural references unless the context clearly dictates otherwise. Thus, for example, reference to “a support material” includes reference to one or more of such materials, “a contaminant” includes reference to one or more of such materials, and “an activation step” refers to one or more of such steps.

As used herein, “activating” refers to any process which results in an increase of binding sites for attachment of the non-living functional group.

As used herein, “biologically-derived” refers to any material which is directly obtained from a biological material such as plant, animal, microbial, or other living organism. Typically, a biological material does not have to be currently living in order to be useful, although living organisms such as microbes can also be useful. Thus, a microbe can be a biologically-derived functional group carrier. Further, grasses, plants, animals, microbes, etc. can be treated as described herein subsequent to harvesting.

As used herein, “bind to” or “bound to” refers to an association of at least two materials which can include chemical covalent bonding, weak force bonding such as ionic, hydrogen bonding or other weak or electrostatic forces, mechanical immobilization, or transformation of at least one of the species. For example, transformation can involve temporary binding where contaminant species are chemically changed through a reaction or exchange of electrons or atoms.

As used herein, “high surface area” is any surface which exceeds a bulk non-porous material by a factor greater than 25. Typically, high surface area materials can have a BET surface area from about 75 m²/g to about 1000 m²/g, and often from 100 m²/g to about 500 m²/g, although exact surface areas can vary among materials. Generally, a higher surface area results in higher treatment flow rates, higher adsorption or binding capacity, higher density of functional groups, and yields, although diffusion may become a rate limiting step.

As used herein, “integrate or integrated” when used to describe an association of multiple chemical species refers to any association of at least two chemical moieties which is temporary. Integrated materials can include, complexed, chelated, reacted by covalent or coordinate bonds, or other associated groups, including association via hydrogen bonding, electrostatic forces, or other weak attractions. The physical and chemical form, lattice, energy, and/or the moieties, SH, OH, etc. present in the structure formed and their coordination or association with various other elements within the integrated functional group can affect the type and strength of the integrated functional group.

As used herein, “mesoporous” refers to a porous structure having average pore diameters between macroporous and microporous features. Mesoporous materials have an average pore diameter from 2 nm to 50 nm. When a material is referred to as mesoporous, the dominant (i.e. more than either microporous or macroporous) pore morphology is mesoporous, although minor portions of the material can also include macroporous and/or microporous features.

As used herein, “selective” refers to a measurable preference for at least one contaminant over other contaminants. Thus selective functional groups may allow association with any number of contaminants. Functional groups can be specific or generic in their selectivity towards contaminants.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition, or are included only in amounts which are small enough so as to have no measurable effect on the composition.

As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

### Embodiments of the Invention

In accordance with the present invention, a modified bioreactor support material having high surface area for removing a contaminant from fluids can include a substrate having a functionalized surface. The functionalized surface can have inorganic or organic non-living functional groups such that the functional groups bind to or chemically alter the contaminant, e.g. chemisorption. Further, the immobilized biomass containing suitable functional groups can be particularly useful with high selectivity and can be regenerated using relatively mild regenerating solutions.

Substrate

Any number of materials can be used as the substrate. Typically, the substrate can be a porous substrate so as to provide increased surface area sufficient to offer high contaminant removal rates per volume of material. However, in some embodiments, a non-porous material can have sufficiently high surface area to act as a suitable substrate. The choice of substrate material can depend on a variety of factors including, among others, availability, performance, and costs. As a very general guideline, suitable substrate materials can include, but are in no way limited to, plastics, zeolites, silicates, activated carbons, starches, lignins, celluloses, plant materials, animal materials, biomaterials, and combinations thereof. In another specific embodiment of the present invention, the substrate can be a mesoporous material.

Plastics can be particularly suitable for applications which require more durable substrates and increased functionality, e.g. tailorability, choice of attachment sites for the functional groups, increased commercial availability, and the like, than conventional activated carbon or silicates like pumice. Suitable plastics for use as the substrate can include, but are not limited to, ABS pellets, nylon pellets, polycarbonate pellets, polyethylene pellets, polyester pellets, polypropylene pellets, polystyrene pellets, PVA pellets, virgin PVC pellets, other plastic pellets, PVC compound, PET, linear low density polyethylene, PVC powder, PVC flexible pellets, thermo plastic granules, polysulfones, and composites or combinations thereof. Additional specific non-limiting examples of suitable substrate materials include high density polyethylene, low density polyethylene, polypropylene, poly(vinyl chloride), poly(vinylidene chloride), polystyrene, polyacrylonitrile, polytetrafluoroethylene, poly(methyl methacrylate), poly(vinyl acetate), cis-polyisoprene, polychloroprene, and combinations thereof.

In one specific embodiment, the substrate can comprise an activated carbon. Non-limiting examples of suitable activated carbons can include magnetic activated carbon, and carbon treated with chemicals such as zirconium and iron. Activated carbons can be formed from a variety of carbon sources such as coal, wood, or other cellulosic materials. Activated carbons are available in a wide variety of configurations. Conventional granular activated carbons (GAC) while useful in some reactor configurations can be susceptible to plugging and may not have the optimal surface area that can be found in pelletized activated carbon (PAC) that is less subject to plugging. Generally, activated carbons can be modified in their surface structure by chemical/heat treatments. In another alternative embodiment, magnetic activated carbons (MAC) can be useful as a substrate material and can be recovered easily by magnetic separation from streams to which it has been added, even from those streams that contain high solids content. Powdered activated carbons have the advantages that the surface structure modification results in higher metal-ion/complex loading capacity and higher adsorption kinetics than that of conventional granular activated carbon.

In another aspect of the present invention, the substrate can further include an inorganic nanopowder in various matrices and configurations. Inorganic nanopowders can provide very high surface areas while also offering control over binding sites for functional groups. In some embodiments, the inorganic nanopowders can be nanomagnetic materials which can be added to substrate materials such as biopolymers and/or plastic carrier materials. Other suitable substrate materials can include structured compositions and structures assembled from various powdered forms of the materials listed herein. For example, mixtures of materials listed herein can be formed to provide a tailored combination of surface area, binding sites, and other factors, e.g. activated carbon and nanopowders can be mixed in various ratios to provide a suitable substrate.

Substrate materials of the present invention can be provided as pellets, powder, granular, extruded, aerogel, or other forms. In addition, the substrate material can have a microporous or macroporous structure. The specific average pore radius and pore volumes can vary considerably among various materials, and can be considered when choosing an appropriate substrate for a specific application.

Activation of the Substrate

In accordance with the present invention, the substrate can be activated prior to forming the functionalized surface. Most substrate materials benefit from an activation step which results in an increase in attachment sites for association of the functional groups, although some non-activated substrates can be useful. Activating the porous substrate can be performed using any treatment which exposes additional binding sites on surfaces of the substrate (whether internal pore surfaces or external surfaces). Non-limiting examples of activation mechanisms can include one or more of the following; heating the substrate, contacting the substrate with an acid such as hydrochloric or sulfuric acid, contacting the substrate with a base, exposing the substrate to ultra-violet radiation, or contacting the substrate with other chemicals such as gluteraldehyde. Various organic chemical treatments can also be suitable such as exposure to gluteraldehyde, bromine, nitrate, etc. Additional non-limiting examples of several specific chemical treatments can include inorganics and organics such as iron, sulfates, sulfides, protamine polymers, amino acids, and acids such as citric, hydrochloric, sulfuric, nitric, and humic.

In one detailed aspect of the present invention, activation of the substrate can include a second activation mechanism. For example, the substrate can be contacted with a coactivation agent selected from the group consisting of iron, sulfates, sulfides, protamine polymers, amino acids, citric acid, hydrochloric acid, sulfuric acid, nitric acid, humic acid, yeasts, proteins, enzymes, biopolymers, and combinations thereof. The primary treatment can be done separately or in conjunction with various plant proteins such as agars, algenates, polypeptides, mixtures and polymers, and plant materials, plant proteins and biopolymers and or living biomaterials. For example, semi-degraded cellulose and sugar structures can be included before using them as treatments to increase the stability of metal specific biopolymers and metal sorption to an activated substrate.

The specific activating steps can depend on the substrate and the desired attachment sites for functional groups. More specifically, the chemical composition of the substrate can largely determine optimal activation mechanisms. Plastics are often best activated by UV and chemical treatments like gluteraldehyde that provides specific contaminant binding sites and/or ‘sticky’ or rougher surfaces for attachment of biological functional groups. Activated carbons and silicates are usually best activated by treatment with acids which amplify the number of binding sites and clean the surface in a manner that enhances binding of biopolymers alone and/or biological functional groups and chemical active sites. Activating increases the density of potential binding sites on the substrate. Although binding sites can vary, most common binding sites can include, but are not limited to, activated groups such as carboxyl, lactone, phenol, ether, pyrone, amino, sulfhydril, hydroxyl, carbonyl, and combinations thereof.

At least one purpose of various additional inorganic and organic activations is to establish a higher density sorption of the specific contaminant binding microbial protein to the substrate and to increase the number of functional binding groups for a specific contaminant. Activation steps can also increase binding of metal contaminants and stabilize the biopolymers, biomaterials, enzymes, and bioactive plant and microbial materials which are actually used specifically for metal binding and/or transformation. In another aspect of the present invention, similar activation steps can allow binding functions to be renewed after the bound and sorbed metals are stripped from the materials. The surface structure of the substrate can be modified by combinations of biochemical, biopolymer, chemical, and other treatments. Further, activation of mesoporous materials can further increase mesoporosity and allow for increased functional group binding sites.

Without being bound by any specific examples, the following provide general guidance regarding particular substrates and currently preferred activations. The choice of activation step can also depend on the target contaminants and conditions of the wastewater stream. Some activation steps can act to remove debris or other interfering groups from a surface, while other activation steps can chemically alter the substrate by leaving activation or binding groups on the substrate surfaces. For example, for selective removal of nitrates, activation of pumice can be effectively accomplished by treatment with hydrochloric acid. In this case, acid activation is dominantly a cleaning and preparation step that would be followed with a biopolymer treatment that would contain live microbes and/or non-living functional groups. Similarly, for selective removal of arsenic, pumice can be preferably activated by treatment with sulfuric acid with a similar subsequent step as mentioned above. In this case, activation is dominantly a chemical change of the surface which leaves sulfate groups which act as binding sites and also enhance subsequent biopolymer, microbe, and biological functional group binding.

Precursor of Contaminant and Functional Group

In accordance with the present invention, a sample contaminant can be associated with a non-living or living biologically-derived functional group to form an integrated group in order to identify suitable candidate functional groups which remove certain contaminants from fluids. Preferably, the functional group can be selective to a specific contaminant. Association of contaminants with a functional group can generally occur via ionic bonding, hydrogen bonding, or other electrostatic interactions, although other associations can be made. In some embodiments, the functional groups can complex, chelate or covalently bond with the contaminant. The integrated group can be particularly useful in designing targeted contaminant removal materials which are highly selective for specific contaminants.

Examples of specific contaminants which can be used in connection with the present invention include, but is not limited to, arsenic, selenium, phosphorous, mercury, cadmium, chromium, manganese, magnesium, zinc, nickel, lead, iron, cobalt, copper, nitrate, cyanide, sulfate, sulfur, silver, gold, and combinations or ions thereof. Thus, this list includes ion compounds of these metals in various oxidation states, e.g. arsenate, arsenite, selenate, etc.

Similarly, the biologically-derived functional group can be extracted or acquired from any number of a variety of biological materials. Of particular interest is the use of biomaterials which readily absorb many common contaminants. Depending on the particular functional groups, binding of various metals can be highly specific to particular metal or general to metals. Non-limiting examples of suitable biomaterials which include useful functional groups can include bio-polymers, proteins, enzymes, lipids, amino acids, vitamins, algae, moss, fungi, grasses, shrubs, bacteria, extracts thereof, and combinations thereof. Biomaterials which are particularly useful as sources for functional groups can include algae, fungi, grasses, seeds, shrubs, bacteria, mosses, and combinations of these materials. Various inorganic materials are required by all living organisms for growth and proper function. Biomaterials typically sorb, bind, and/or transform various metals and other inorganics. This property of biomaterials can be exploited by incorporation into the present invention as described herein. In one specific embodiment, the non-living functional group can include a bio-polymer selected from the group consisting of alginates, polypeptides, gels, agars, yeasts, starches, lignins, microbial extracts, plant materials, animal materials, bacteria, enzymes, proteins, and combinations thereof. Other biomaterials which have suitable functional groups can include plant refuse (e.g. grass, shrubs, corn stalks, chopped trees, sawdust, leaves, malinga olifera seeds, roots, fruits, and the like), animal remains (e.g. bones, skin, organs, etc.), bacteria and algae (e.g. Cyanobacteria, Diatoms, Alcaligenes sp., Escherichia sp., Pseudomonas sp., Desulfovibrio sp., Shewanella sp., Bacillus sp., Thauera sp., P. putida, P. stutzeri, P. alcaligenes, P. pseudoalcaligenes, P. diminuta, Xanthomonas sp. including X. (Pseudomonas) maltophilia, Alc. Denitrificans, various Bacillus species Bacillus species that are versatile chemoheterotrophs including B. subtilis, B. megaterium, B. acidocaldarius, & B. cereus, Cellulomonas and Cellulomonas Fermentans, various sulfate reducing bacteria including Desulfobacter, Desulfobulbus, Desulfomonas, Desulfosarcina, Desulfotomaculum, Desulfurocococcus, Desulfotomaculum, and Desulfuromonas species, Nitrosomonas, Nitrobacter, Rhodobacter, Thiobasillus, and Geobacter species, E. coli, and various Achaea bacteria and combinations of these bacteria, kelps, seaweeds; and various algal species used to produce agars and alginates as well as specific binding sites for various metals), fungus, water plants (e.g. varieties of azolla, water hyacinths, salvinia, wolffia or duckweeds, moringa sp.), moss (Pleurocarpous and Acrocarpous mosses including Sphagnum mosses such as Sphagnum affine liverworts including Scapania paludicola), high oil-producing biomaterials, and the like. Many fungus, grass and shrub species have proteins and enzymes that can be useful in metal binding and transformations; such as denitrification and cyanide degradation which can be readily identified based on the disclosure herein. Plant and algal cellulose and lignin structures are particularly useful in water treatment following pretreatment with acids, e.g. HCl, H₂SO₄, and HNO₃, and bases, e.g. sodium hydroxide, detergents such as sodium dodecyl sulfate, to create receptive surfaces for coatings containing high concentrations of metal reactive materials and surface metal binding sites.

In connection with the present invention, enzymes and proteins can also be useful alone or in combination with these microbes. Many of the previously listed plant, bacteria and other materials include active proteins and/or enzymes which are the primary binding centers for removal of the contaminants. However, such proteins and/or enzymes can be isolated or independently identified and can include, but are not limited to, oxidizing and reducing enzymes, electron transport components, dimetallic phosphatases, DNase I related nucleases/phosphatases, dioxygenases, and metalloproteins, and proteins, including bacterial and plant cell components and biopolymers. Similarly, chemical and pressure treatments can affect the number of available binding sites. For example, the bioreactor support material can be subjected to an increased pressure sufficient to induce an increase in available functional group binding sites.

Generally, inorganic and organic non-living functional groups can be attached on a substrate surface. Non-limiting examples of inorganic functional groups can include copper, zirconium, iron, aluminum, and combinations thereof. In one detailed aspect, the functional groups can include a mixture of biomaterial and inorganic materials.

In some embodiments of the present invention, non-living functional groups can be used, including groups which are isolated from living materials such as microbes, bacteria, algae, etc. In other embodiments, living functional groups can be used. Alternatively, the functional groups of the present invention can include combinations of both living and non-living functional groups.

Biologically-derived functional groups can include living materials which are maintained as living organisms during use of the final support material. In particular, a microbial population can be cultivated which is designed for a particular wastewater stream. Populations of microbes can be provided either from known sources or a portion may be obtained by cultivating native microbes. Frequently, a combination of native microbes and microbe populations known to have selectivity for a particular contaminant can be effective. The native microbes can contribute additional population stability during use, while select introduced microbes can further augment removal of specific contaminants.

Any candidate microbial population can be monitored to identify contaminant selectivity. The microbial population can also be tested using sample water in order to verify persistence of the population over time. Although variations in microbe population naturally occur during use, optimization of a target microbial population for a particular water stream can minimize large fluctuations in performance and/or microbe population. A candidate microbial population can be readily profiled such as by nucleic acid extraction and profiling. Such profiling can also be performed during use of the final reactor support material in order to monitor the microbial population. If the population fluctuates beyond predetermined levels the material can be either replaced, or a supplemental microbe population or conditions such as nutrients can be adjusted in order to return the population to a desired level.

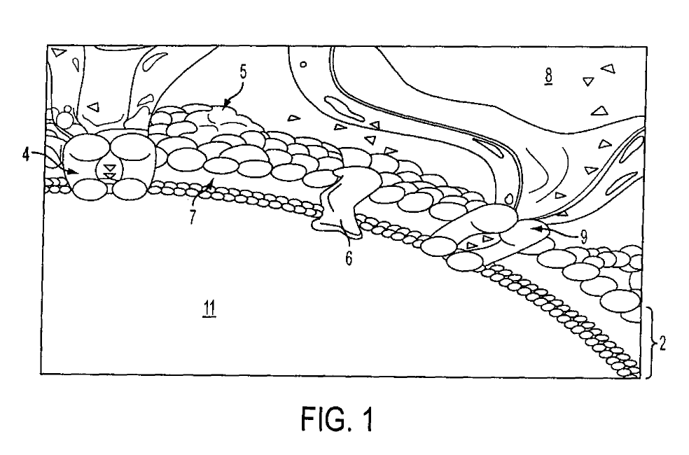

A suitable functional group source material can be mixed with a sample contaminant. The functional group source material can be modified or unmodified. Specifically, in some cases, biomaterials such as moss, algae, bacteria, etc. can be mixed with a sample contaminant without any modification. These and other biomaterials include functional groups which naturally associate with a sample contaminant. FIG. 1 illustrates metals associating with a microbial cell wall 2 which shows a number of possible mechanisms for associating with a contaminant, e.g. precipitation, metal binding proteins, incorporation into cell walls, oxidizing enzymes, reducing enzymes, and the like. A metal precipitator 4 by biological material (e.g. H₂S, CO₂, O₂, etc.) can bind metals via precipitation. A metal binding protein 5 can bind metals at the cell wall 2. Metals can be incorporated directly into modified cell wall segments 6 across the cytochrome system 7. Metal gas 8 or solid can be oxidized via metal oxidizing enzymes 9 upon uptake. These mechanisms and other can also be found inside the boundaries of the cell wall 2. For example, metal retention in cellular traps, metal transforming enzymes, metal reducing enzymes, metal binding proteins, metal oxidizing enzymes, metal oxidizing/reducing enzymes, and nucleic acids can all act on various metals to bind, transform or otherwise render immobilized or harmless such contaminants according to known mechanisms. FIG. 1 illustrates a microbial cell surface 2 and interior 11 showing various metal sorbing, binding, and transforming mechanisms available for immobilization of a contaminant in the biomaterial matrix. Many of the above biomaterials sorb and bind metals over a wide range of conditions, e.g. a pH range of 3 to 9.5. The biomaterial can then be disruptively agitated, chemically treated, extracted, or otherwise treated so as to remove the functional groups having the sample contaminants associated therewith. The specific functional groups can be optionally isolated and recovered using conventional methods such as fractionation to isolate proteins/enzymes, or the like. The result is an integrated functional group having a sample contaminant associated with a non-living functional group. Optionally, the recovered functional groups can be incorporated into a biopolymer matrix resin as a protective environment. For example, alginate can be suitable for many functional groups as a matrix resin material, although other materials can also be used.

In another alternative embodiment, the functional group source material, such as a biomaterial, can be conditioned or optimized for specific contaminants. Specifically, preparations of bioploymers, cellular biomaterials, and/or enzymes can be prepared by adaptation and culturing or incubation of various microbial populations with concentrations of the target metal or metalloid. Once these materials are carried through the adaptation/optimization process, biopolymers and cellular materials can be obtained from microbial cells by various extraction methods. For example, extraction using cell disruption and partial separation of cellular components such as, but not limited to, mechanical disruption, lysozyme, detergents and combinations thereof can be used.

Association of Functional Groups with Substrate

Once the desired functional groups have been identified, the functional group can be attached to a suitable substrate. This can typically involve contacting the functional group in a liquid medium with the substrate. Typically, the non-living and/or living functional groups can be attached to the porous substrate via hydrogen, ionic, or covalent bonding. In some embodiments, activating the substrate and attaching the functional groups can be performed substantially simultaneously. The resulting modified bioreactor support material can have a functionalized surface including a plurality of inorganic and/or organic functional groups. Although exact functional group densities can vary considerably, the functionalized surface can include from about 200 to 30,000 of the functional groups per mm³, in some cases from about 1000 to 5,000, and in some cases from about 10,000 to about 30,000 groups per mm³. Further, in some applications, the functional group density can be relatively low, e.g. from 10 to about 50 units per mm³. The functional group densities can vary considerably depending on the particular substrate and functional group combination, as well as the specific method of activation and attachment. It is emphasized that actual densities of these functional groups can vary considerably and can depend upon the nature of the treated surface, materials extracted, and the method of immobilization.

When at least a portion of the biologically-derived functional groups are living functional groups, the target microbial population can be sufficient to inhibit growth of non-target microbes. In particular, the living functional groups such as those associated with microbes can form a protective layer or biofilm which inhibits growth of non-target or foreign microbes which can otherwise clog mesopores and/or reduce contaminant removal efficiencies. Attachment of living functional groups can occur in a similar manner to non-living functional groups. Further, living functional groups typically readily attach to a variety of substrates by exposing the microbes to surfaces of the activated substrate. Activation of the substrate can be done to increase binding sites using the same approaches described previously. A gel or media of an organic composition of nutrients, aerobic and/or anaerobic microbes, proteins, enzymes, inorganic additives, and/or nanoparticles can be prepared. Inorganic activation using acids and additives such as iron hydroxides, zirconium, and the like can help to enhance binding to a substrate surface and can aid in configuring the contaminant active groups to effectively associate with the contaminant. Various modes of microbe-metal-protein interaction can include metal-, ligand-, and enzyme-complexes that can serve as electron donors or acceptors and through close association can increase live microbial—contaminant interaction, binding, and/or transformation.

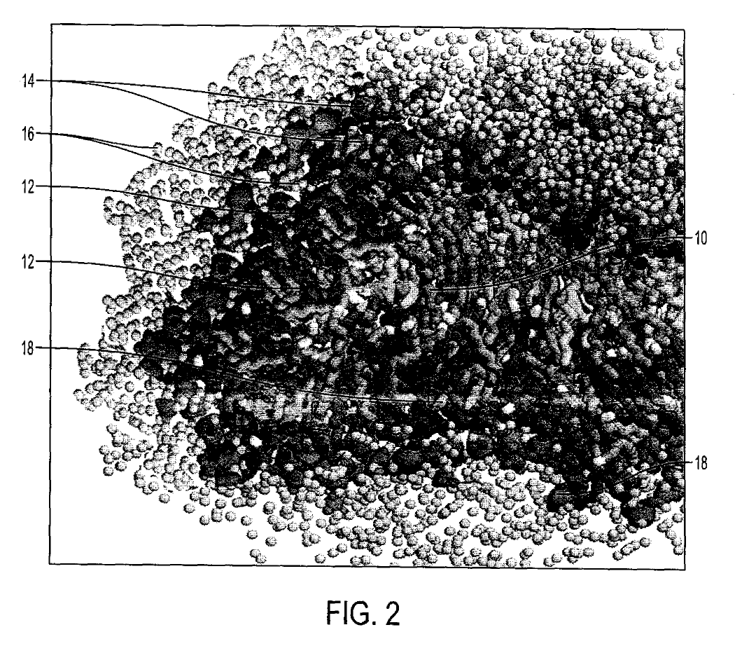

FIG. 2 illustrates a porous support matrix 10 having a combination of functional groups associated therewith. The illustrated surfaces are greatly magnified such that the overall porous structure is not evident. As can be seen, metal binding functional groups 14 and/or microbes 18 can be embedded into and on a support matrix material having activated support sites 12. Each contaminant binding group can be associated, penetrated or embedded into the matrix material to varying degrees, depending on pretreatment, activation, concentration of functional groups, porosity of the substrate, incubation pH, exposure time, and the like.

In yet another optional embodiment of the present invention, a secondary treatment can further modify the functional group and binding capacity of the bioreactor support materials. For example, addition of a metal pretreatment 16 can increase contaminant uptake. Zirconium and copper can be particularly suitable and have shown to increase removal of arsenic from contaminated liquids. Further, optional catalytically active materials can be incorporated into the substrate or the functional groups attached to the surface of the substrate. These materials can act to catalyze reaction of contaminants into a form which is less harmful and/or more readily recovered, e.g. precipitated, etc. Non-limiting examples of suitable catalytically active metals can include iron, magnesium, manganese, other metals, alloys thereof, and the like. Various modes of metal-protein interaction can include metal-, ligand-, and enzyme-bridge complexes. Metals can serve as electron donors or acceptors, Lewis acids, and/or structural regulators. Those that participate directly in a catalytic mechanism usually exhibit anomalous physicochemical characteristics reflecting their interior structural location. Carboxypeptidase A, liver alcohol dehydrogenase, aspartate transcarbamoylase and alkaline phosphatase exemplify the different roles of metals in metalloenzymes.

The sample contaminant can be removed from the modified bioreactor support material either before or after shipment to an end user. In particular, most sample contaminants can be removed through flushing and subsequent recovery in a smaller volume or as a concentrated precipitate.

Applications of Support Material

The modified bioreactor support material can then be used in any number of contaminant removal scenarios. The material of the present invention can be useful in wastewater treatment, culinary water treatment, treatment of industry effluent, recovery of valuable or precious metals, or any other application which benefits from removal of specific contaminants from a contaminated fluid. Removal using the materials of the present invention can include contacting the contaminated fluid having a contaminant therein with the modified bioreactor support material under conditions such that the contaminant is bound to the substrate or is chemically altered thereby. The support material can be used in bulk, incorporated into columns, or placed in tanks where contaminated fluids are then passed through or mixed with the material. Upon reaching a predetermined uptake threshold, the support material can be replaced or reconditioned to eliminate the removed contaminants.

In another alternative embodiment, the support materials of the present invention can be incorporated into a biosensor or biodetector based on the number or percentage of functional groups occupied by a contaminant. Such devices can operate on well known principles such as, but not limited to, on-site conductivity tests, color change dyes, or the like. For example, proteins with a high specificity for arsenic or other contaminant can be made to fluoresce or induce a color change. Alternatively, the support material can be lab tested after exposure to a fluid to be tested.

The support materials of the present invention can be regenerated or recycled once contaminant removal falls below a predetermined level. Regeneration can be accomplished by removing the contaminants from the material, e.g. by back flushing or flushing with a suitable solvent or weak acid. Although long-term stability can vary largely depending on the materials, as a general rule, plastic substrate can provide for an increased number of regeneration cycles and longer service life, while more fragile substrates can provide better performance with generally lower service life. However, specific combinations of substrates and functional groups can be optimized and tested to increase performance under particular contaminants, wastewater conditions, and other usage parameters.

### EXAMPLES

### Example 1

### General Examples

10, 25, 50, 75 and 100 milligrams of proteins, enzymes, biomaterials, prepared from microbial, plant, and/or animal biomaterials were used for mixing with various other materials such as combining in solutions containing low 0.1 to 1 M solutions of various iron, sulfates, sulfides, protamine polymers, amino acids, citric acid, hydrochloric acid, sulfuric acid, nitric acid, humic acid, yeasts, proteins, enzymers, other biopolymers and inorganic and organic nano-materials, For example, a solution was created containing 50 mg of microbial biomaterials isolated by disrupting microbial cells (mixture of Pseudomonas sp. Pseudomonas, Burkholderia, Bacillus sp., sulfate reducing bacteria (e.g. desulfovibrio), E. coli, Alcaligenes and Cellulomonas sp.) using enzymes (e.g. lysozyme) and detergents (e.g. SDS, Tween, Triton X, and CHAPS). These treatments were followed by salt precipitation designed to remove a specific range of proteins and enzymes. The materials were stored in normal (0.85%) saline at 4° C. to −20° C. until use.

These materials were then combined with alginates, polypeptides, gels, agars, yeasts, starches, lignins, other non-living microbial, plant, and animal materials by slow rpm mixing at 4° C. to 60° C. and then attached to or mixed with plastics, zeolites, activated carbons, silicates, activated carbons, activated and non-activated plant and animal materials, and other biomaterials such as bone, chitin, etc., activated plant material through hydrogen, ionic, or covalent bonding, for example with gluteraldehyde.

Attachment was by pretreating existing support materials such as carbon, plastic, etc. materials with acids, bases, organic solvents, or UV treatments. For example activation was achieved by treating the substrate with an amount of 1M hydrochloric acid or sulfuric acid to cover the substrate, washing with water, and then mixing with various concentrations of a combination of the above solutions. In another case these solutions were combined with precursor solutions of plastics, urethanes, alginates, or polysulfones and hardened with solvents and/or various ionic solutions and/or crosslinking components, i.e. magnesium chloride, and/or sequentially with other divalent and trivalent ions in 1M solution to polymerize the solution. Biomaterials were obtained commercially from companies such as Fisher, Aldrich, Sigma, or prepared in the laboratory from suitable starting materials.

### Example 2

### Example of Selenium and Cyanide Removal

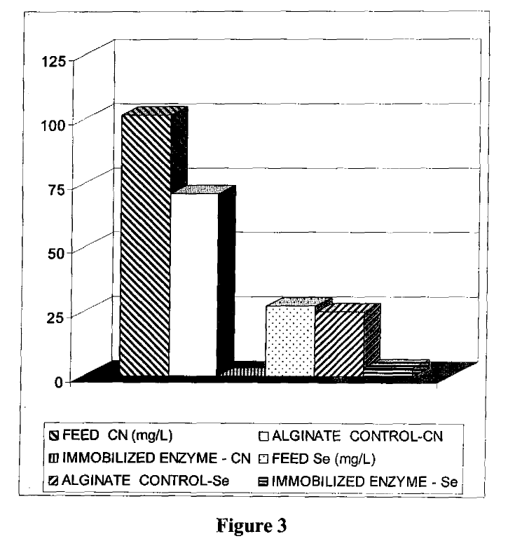

500 micrograms of selected microbial biomaterials were prepared by disrupting microbial cells (e.g. Pseudomonas, Burkholderia, and Bacillus sp.) followed by ammonium sulfate salt precipitation of selected proteins and enzymes. These microbial materials were immobilized in low concentration calcium alginate solution through polymerization in a 1M ionic solution at 4° C. An actual mine wastewater containing selenium and cyanide at the indicated starting concentrations was treated in an up-flow column reactor containing the alginate-immobilized biomaterials. The reactor used a retention time of 24 hr at ˜20 C. Results are illustrated in FIG. 3 and analysis was by ICP.

### Example 3

### Example of Cyanide Removal

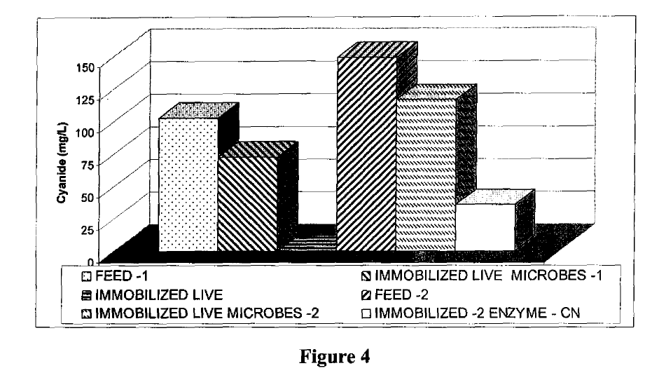

500 micrograms of selected microbial biomaterials were prepared by disrupting microbial cells (e.g. Pseudomonas, Burkholderia, and Bacillus sp.) followed by salt precipitation of selected proteins and enzymes via ammonium sulfate fractionation. These microbial materials were immobilized in low concentration calcium alginate solution through polymerization in a 1M ionic solution at 4 C. A characterized microbial population of principally Pseudomonas microbes at a concentration density of ˜1×10⁹ was used to compare live microbial oxidation of cyanide with enzymatic cyanide oxidation. The live microbes were incorporated into a biopolymer matrix in a similar manner as were the enzymatic materials and tested along with control biopolymers to examine cyanide removal. An actual mine wastewater at pH 10.6 containing free and complexed cyanides at the indicated starting concentrations was treated in a column reactor containing the alginate-immobilized biomaterials. The reactor used a retention time of 24 hr at ˜20 C. In this example, cyanide toxicities to living microbes and lower oxidation rates are evident. Enzyme activity is more dependent upon contaminant concentration than are live microbes and are not affected by higher concentrations of cyanide as are the live microbes. Cyanide analysis was by distillation and the results are shown in FIG. 4.

### Example 4

### Example of Various Metal Removals

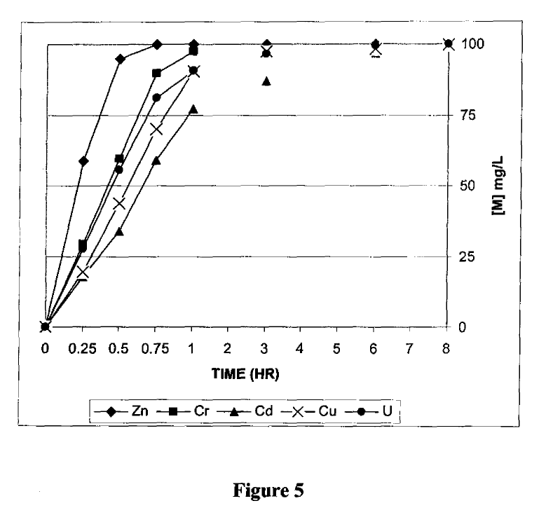

500 micrograms of selected microbial biomaterials were prepared by disrupting microbial cells (e.g. Pseudomonas, E. coli, sulfate reducing bacteria, Alcaligienes, and Bacillus sp.) followed by ammonium sulfate salt precipitation of selected uncharacterized proteins and enzymes. Somewhat different microbial starting mixtures, thus different protein and enzymes were used for the generation of each of the curves. These microbial materials were tested in a normal (0.85%) saline solution at pH 7 using 100 mg/L of each of the various metals. The solution of microbial biomaterials and metals were slowly mixed at ˜20° C. for the time indicated and sampled as indicated by the points on the different curves. Analysis was by ICP and results are shown in FIG. 5.

### Example 5

### Example of Selenium and Arsenic Removals

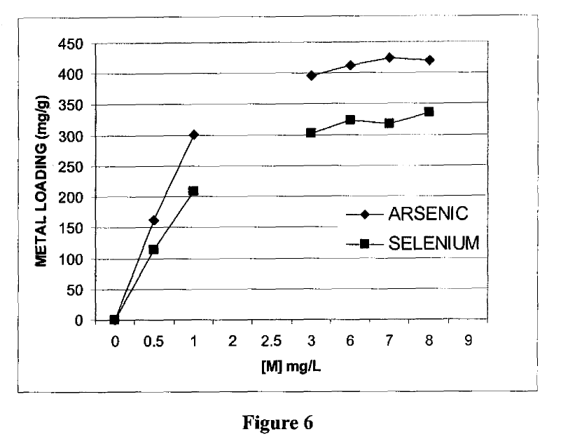

500 micrograms of selected microbial biomaterials were prepared by disrupting microbial cells (e.g. Pseudomonas, sulfate reducing bacteria, and Bacillus sp.) followed by salt precipitation of selected proteins and enzymes. Different microbial starting mixtures, thus different protein and enzymes were used for the generation of each of the curves (e.g. Pseudomonas sp. Pseudomonas, Burkholderia, Bacillus sp., sulfate reducing bacteria (e.g. desulfovibrio) and E. coli), and combinations of biomaterials extracted from these microbial species. These microbial materials were tested in a normal saline solution at pH 7 using 5 g/L of each of the various metals. The solution of microbial biomaterials and metals were slowly mixed at ˜20° C. for the time indicated and sampled as indicated by the points on the different curves. Analysis was by ICP with the results shown in FIG. 6.

### Example 6

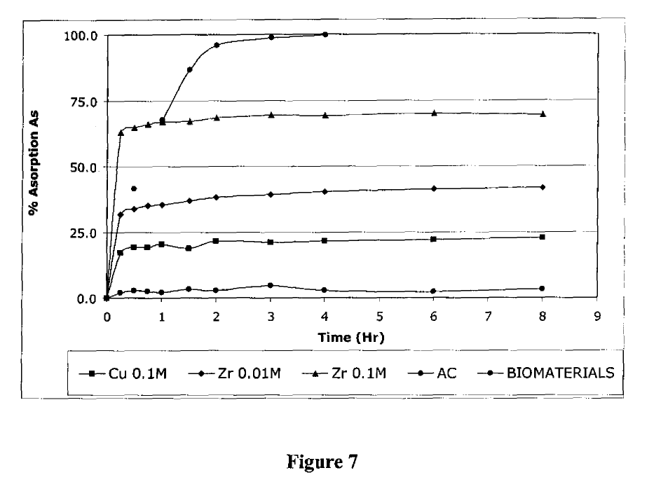

Comparison of arsenic removal by inorganics bound to a modified activated carbon material and biomaterials bound to modified powered activated carbon. 1000 micrograms of selected microbial biomaterials were prepared by disrupting microbial cells (e.g. various sulfate reducing bacteria and Pseudomonas sp.) followed by salt precipitation of selected proteins and enzymes. The microbial materials were combined with 100 micrograms of iron nanoparticles and 100 micrograms of protamine solution. The binding of arsenic was enhanced by pretreatment of the carbon with sulfuric acid followed by a water wash and drying. The biomaterials, iron nanoparticles, and protamine solution were mixed together at ˜20° C. for 5 minutes followed by a 5 minute mix with a 0.05% alginate solution then combined with the carbon and mixed for 5 min again at ˜20° C. The treated carbon was then put in a normal saline solution at pH 7 containing 5 gm/L arsenic. The solution of microbial biomaterials and metals were slowly mixed at ˜20° C. for the time indicated and sampled as indicated by the points on the different curves. The two different inorganics: A-copper and B-zirconium were ionically bound to modified magnetic activated carbon at the concentrations indicated and mixed with an arsenic solution containing 5 gm/L arsenic. Analysis was by ICP. Arsenic adsorption over time for the identified materials is shown in FIG. 7.

### Example 7

### Bio-Oxidation of Repeated Cyanide Additions

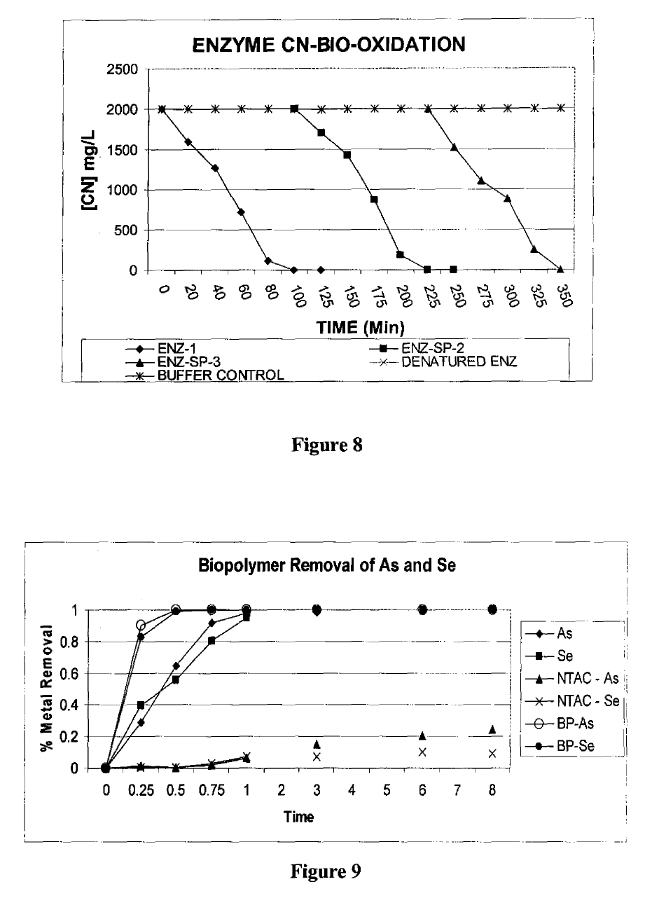

Example of repeated additions of cyanide followed by removal using 1000 milligrams of selected microbial biomaterials was prepared by disrupting microbial cells (e.g. Pseudomonas, Bacillus, and Burkholderia sp.) followed by salt precipitation of selected proteins and enzymes. These microbial materials were immobilized with an electron acceptor in low concentration calcium alginate solution through polymerization in a 1M ionic solution at 4° C. An actual process solution at pH ˜9.5 containing free cyanide at the indicated starting concentrations was treated in a column reactor containing the alginate-immobilized biomaterials. The reactor used a retention time as shown at ˜20° C. The three curves demonstrate that the immobilized cyanide degrading enzyme-electron acceptor complex immobilized in alginate was stable and degraded three separate batch solutions of high concentration free cyanide solution with the change in cyanide concentration over time shown in FIG. 8.

### Example 8

This example included treating activated carbon and binding the enzyme to a granular activated carbon in a manner similar to that described for FIG. 7. The results were very similar to that shown in FIGS. 5, 7 and 9.

### Example 9

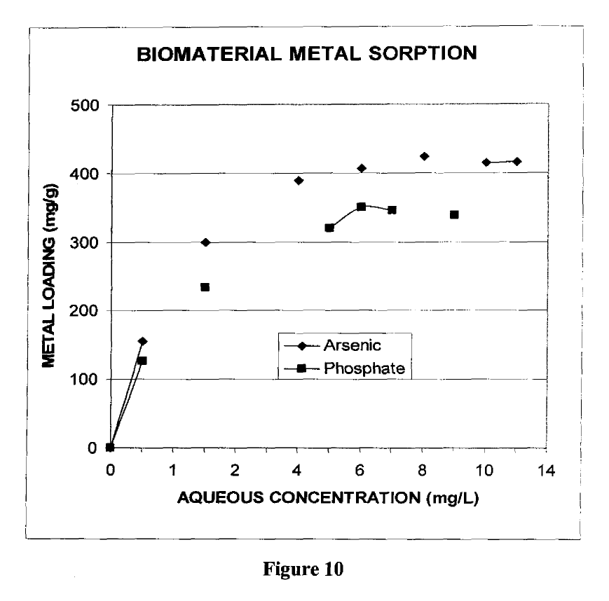

Tests were completed at circumneutral pH at ˜24° C. FIG. 8 shows metal removal over time using 50 mg biopolymer materials mixed with 1 gm/L metal. FIG. 9 shows metal removal over time using 50 mg biopolymer materials tested with 1 gm/L metal alone, bound to activated carbon, and activated carbon controls —As—Se—As— and Se Binding/Sorption of metals in solution without the activated carbon carrier; NTAC—Non-treated Activated Carbon—As and Se test controls; and BP—As & Se—Activated carbon as a support for As or Se binding biopolymers. FIGS. 6 and 10 show Langmuir isotherms using 25 mg or metal specific biopolymer materials with varied concentrations of metals. FIG. 7 illustrates sorption and binding of arsenic by activated carbon alone, with 0.1M inorganic A (i.e. copper) treated activated carbon, 0.1M inorganic B (i.e. zirconium) treated activated carbon, and activated carbon treated with 50 mg biopolymers.

Various testing suggests that some of these additions are additive and some stabilize the biopolymer materials on the activated carbon sufficient to allow the bioreactor support material to be regenerated or recycled.

It is to be understood that the above-referenced arrangements are illustrative of principles of the present invention. Thus, while the present invention has been described above in connection with the exemplary embodiments of the invention, it will be apparent to those of ordinary skill in the art that numerous modifications and alternative arrangements can be made without departing from the principles and concepts of the invention as set forth in the claims.

What is claimed is:

1. A modified bioreactor support material having high surface area for removing a contaminant from fluids, comprising a substrate having a functionalized surface, said substrate being activated prior to forming the functionalized surface and said functionalized surface having biologically-derived functional groups, such that the functional groups bind to or chemically alter the contaminant.

2. The material of claim 1, wherein the functionalized surface includes from 200 to 30,000 of the biologically-derived functional groups per mm³.

3. The material of claim 1, wherein the substrate comprises a member selected from the group consisting of plastics, zeolites, silicates, activated carbons, starch, lignins, celluloses, plant materials, metals, animal materials, biomaterials, and combinations thereof.

4. The material of claim 3, wherein the substrate comprises a member selected from the group consisting of high density polyethylene, low density polyethylene, polypropylene, poly(vinyl chloride), poly(vinylidene chloride), polystyrene, polyacrylonitrile, polytetrafluoroethylene, poly(methyl methacrylate), poly(vinyl acetate), cis-polyisoprene, polychloroprene, and combinations thereof.

5. The material of claim 3, wherein the substrate comprises an activated carbon.

6. The material of claim 1, wherein the substrate is an inorganic material.

7. The material of claim 1, wherein the substrate is a mesoporous substrate.

8. The material of claim 1, wherein the substrate further comprises an inorganic or organic material associated with a nanopowder.

9. The material of claim 1, wherein the biologically-derived functional group is a non-living functional group selected from the group consisting of bio-polymers, proteins, enzymes, lipids, amino acids, vitamins, algae, moss, fungi, grasses, shrubs, bacteria, extracts thereof, and combinations thereof.

10. The material of claim 9, wherein the non-living functional group is a bio-polymer selected from the group consisting of alginates, polypeptides, gels, agars, yeasts, starches, lignins, microbial extracts, plant material, animal materials, and combinations thereof.

11. The material of claim 1, wherein the functionalized surface includes living functional groups.

12. The material of claim 11, wherein the functionalized surface further includes non-living functional groups.

13. The material of claim 1, wherein the functional group is selective to a specific contaminant.

14. The material of claim 13, wherein the specific contaminant is selected from the group consisting of arsenic, selenium, phosphorous, mercury, cadmium, chromium, manganese, magnesium, zinc, nickel, lead, iron, copper, nitrate, cyanide, sulfate, and combinations thereof.

15. A method for making a modified bioreactor support material for the removal of a contaminant from fluids, comprising:
activating a substrate to expose binding sites; and
attaching a biologically-derived functional group to the substrate.

16. The method of claim 15, wherein activating includes one or more of heating the substrate, contacting the substrate with an acid, contacting the substrate with a base, exposing the substrate to ultra-violet radiation or contacting the substrate with a gluteraldehyde.

17. The method of claim 15, wherein activating the substrate includes a second activation mechanism.

18. The method of claim 17, wherein the second activation mechanism includes contacting the substrate with a coactivation agent selected from the group consisting of iron, sulfates, sulfides, protamine polymers, amino acids, citric acid, hydrochloric acid, sulfuric acid, nitric acid, humic acid, yeasts, proteins, enzymes, and combinations thereof.

19. The method of claim 15, wherein the activating increases a density of the binding sites on the substrate, said binding sites including activated groups selected from the group consisting of carboxyl, lactone, phenol, ether, pyrone, amino, sulfhydril, hydroxyl, carbonyl groups, and combinations thereof.

20. The method of claim 15, wherein the biologically-derived functional groups are attached to the porous substrate via hydrogen, ionic, or covalent bonding.

21. The method of claim 15, wherein the attaching and activating are performed substantially simultaneously.

22. The method of claim 15, wherein the specific contaminant is selected from the group consisting of arsenic, selenium, phosphorous, mercury, cadmium, chromium, manganese, magnesium, zinc, nickel, lead, iron, copper, nitrate, cyanide, sulfate, and combinations thereof.

23. The method of claim 15, wherein the biologically-derived functional groups include non-living functional groups selected from the group consisting of bio-polymers, proteins, enzymes, lipids, amino acids, vitamins, algae, moss, fungi, grasses, shrubs, bacteria, extracts thereof, and combinations thereof.

24. The method of claim 15, wherein the biologically-derived functional groups include living functional groups.

25. The method of claim 24, wherein the living functional groups are obtained by cultivating a microbial population, monitoring contaminant selectivity of the microbial population, and designing a target microbial population for selective removal of the contaminant to form at least a portion of the biologically-derived functional groups.

26. The method of claim 25, wherein subsequent to the step of attaching, the target microbial population is sufficient to inhibit growth of non-target microbes.

27. The method of claim 15, wherein the substrate comprises a member selected from the group consisting of plastics, zeolites, silicates, activated carbons, starch, lignins, celluloses, plant materials, metals, animal materials, biomaterials, and combinations thereof.

28. The method of claim 15, wherein the substrate is a mesoporous substrate.

29. The method of claim 15, wherein the biologically-derived functional group is identified by associating a sample contaminant with a candidate functional group source material to form an integrated functional group and monitoring contaminant removal rates.

30. A method for the removal of contaminants from a contaminated fluid comprising:
contacting the contaminated fluid having a contaminant therein with the material of claim 1, said contacting occurring under conditions such that the contaminant is bound to the substrate or is chemically altered thereby.

31. The method of claim 30, wherein the biologically-derived functional groups include living functional groups.

32. The method of claim 31, supplementing the living functional groups during use in order to maintain a predetermined microbial population sufficient to prevent substantial loss of contaminant removal performance.

33. The method of claim 31, wherein the living functional groups exhibit a microbial population sufficient to inhibit growth of foreign microbes.

34. The method of claim 31, wherein the material is recycled once contaminant removal falls below a predetermined level by removing the contaminants from the material and repeating the step of contacting.
